(19) United States
(12) Patent Application Publication
(10) Pub. No.: US 2003/0139667 A1
(43) Pub. Date: Jul. 24, 2003

(54) Tissue viability/health monitor utilizing near infrared spectroscopy

(76) Inventors: Mark D. Hewko, Manitoba (CA); Lorenzo Leonardi, Manitoba (CA); Michael G. Sowa, Manitoba (CA)

Correspondence Address: Ade & Company, 1700 360 Main Street, Winnipeg Manitoba, R3C 3Z3, CA

(21) Appl. No.: 10/204,844

(22) PCT Filed: Apr. 12, 2001
(86) PCT No.: PCT/CA01/00585

(30) Foreign Application Priority Data
Apr. 13, 2000 (US) 60196914

(51) Int. Cl. A61B 5/05

(52) U.S. Cl. 600/410

(57) ABSTRACT

An apparatus for the evaluation of tissue parameters in the visible and near infrared as related to tissue status is presented. The apparatus comprises a light source capable of illuminating tissue in the visible and near infrared spectral region. The tissue absorbs some of the light while a large portion of the light is diffusely scattered within the tissue. Scattering disperses the light in all directions with a fraction of the scattered light penetrating into the tissue and remitted back out to the surface. The remitted light is collected by a detection system capable of dispersing the light into its wavelength components. The light can be collected using single or multiple fiber optic probes entering into a dispersive wavelength selection devices in which the dispersed light is detected using a photon detecting device in a spectroscopic milieu. Likewise, the remitted light can be detected in an imaging fashion using a non-dispersive wavelength selection and imaging optical system. The remitted light detected from the tissue contains unique spectral information related to the health status of the tissue. The acquired spectra and images are displayed in near real time on a display in such a manner to characterize the health status of the tissue.

### FIELD OF THE INVENTION

The present invention relates generally to the field of medical devices. More specifically, the present invention relates to a device that non-invasively or with minimal invasion to the body can be used to determine the viability, heath or status of tissue by using visible and near infrared light.

### BACKGROUND OF THE INVENTION

The present and accepted standard for determining the status of tissue relies on visual inspection of the tissue. Based on the surface appearance of the tissue, medical personnel will make an assessment of the tissue and proceed to a course of action or treatment. Visual inspection of tissue is central to many areas of clinical medicine, and remains a cornerstone of dermatology, reconstructive plastic surgery, and in the management of chronic wounds, and burn injuries. For example, in plastic surgery, it is extremely important to assess the status of the tissue prior to surgery, during surgery and following surgery. Detection of complications or tissue compromise before the onset of irreversible tissue damage is paramount. Early detection of tissue compromise following surgery enables a more effective course of intervention to be taken in order to salvage tissue which is at risk of failing. The monitoring of tissue viability or status during and following surgery ensures the efficacy of surgical procedures and non-surgical means of intervention can be determined prior to irreversible damage to the tissue. Unfortunately, visual manifestations of tissue compromise generally become apparent several hours after the onset of the complication. Thus, current clinical assessment methods based on visual examination of the tissue provide an indication of tissue compromise well after the onset of the problem. This delays possible corrective action, which in turn impacts the clinical outcome of the affected tissue. Poor blood supply to the extremities is a common problem among the elderly and diabetic populations. Poor peripheral circulation is the leading cause of amputation in these populations. Poor peripheral blood supply is a major underlying contribution in persistent or chronic wounds of the lower legs and feet. These wounds are difficult to heal and can become infected and gangrenous if not assessed and treated as early as possible. Clinical evaluation of thermal injuries is made to determine if the standard wound care practises will be sufficient to heal the injury or whether there is the need for surgical intervention. The course of action based on visual assessment of the injury is generally made two to three days after the injury and the initial visual inspection. Even with this delayed evaluation the assessment of the injury is only slightly better than the initial guess.

The prior art teaches a number of devices intended to assess tissue viability or status, as discussed below.

Laser Doppler Flowmetry is used to estimate blood flow in the skin. The method has the appeal of an easy-to-use instrument that is minimally invasive. The instrument collects a profile of Doppler shifted wavelengths, which it then fits to a velocity distribution. The relationship between the Doppler profile and the velocity distribution derived for tissue is based on two major assumptions: (1) photons are randomly scattered by the tissue medium; and (2) photons undergo a single collision event before capture by the detector. Based on these assumptions, the fit of the Doppler profile to the velocity distribution provides the rms velocity of the particles that are moving within the tissue that is being probed by the laser light. Anything that perturbs the laser Doppler profile will affect the calculated rms velocity (laser Doppler flux). Thus the instrument is extremely sensitive to motion, be it motion of the probe or motion of the subject. Furthermore, the major drawback to laser Doppler is the enormous variation in the laser Doppler flux from comparable sites between subjects, from different sites in the same subject, and even from the same site in the same individual at intervals of minutes, hours and days. Also, the apparatus attempts to determine the blood flow and makes no endeavour in the assessment of oxygen delivery or utilization in tissue.

Fluorescence dyes can be used to determine the extent of blood perfusion in tissue and vessels. The method involves injecting a dye into the systemic circulation. The dye is then carried to the site of interest by the blood stream. The area of interest is illuminated with light of a suitable wavelength to excite dye fluorescence. If fluorescence is detected, the site is receiving a supply of blood. If only weak fluorescence or no fluorescence is measured, the site is not receiving an adequate supply of blood. The method has demonstrated success in qualitatively assessing blood flow and the extent of perfusion in compromised tissue. However, this method is invasive as it involves the injection of a dye. Furthermore, the extended washout times of the dye limits the frequency with which these methods can be applied to the site of interest. Again, this method was primarily used to measure perfusion, which in turn is used indirectly to assess the status of the tissue.

Transcutaneous Oxygen Pressure Measurement (TCOM) consists of placing a heated oxygen specific electrode on the skin to measure the oxygen diffusing across the skin. The hot TCOM probe is generally not placed directly on the compromised tissue to avoid further injury to the tissue. Oxygen delivery to the compromised tissue is inferred by measuring the healthy tissue surrounding the compromised tissue. The heating of the tissue beneath the electrode increases tissue perfusion. Thus, the oxygenation of the heated, healthy tissue must be extrapolated to give an indication of the oxygen delivery at a neighbouring injured or compromised site. TCOM, when combined with a standard measurement protocol, is an effective and non-invasive means of identifying tissues and wounds receiving inadequate levels of oxygen. However, the TCOM measurement protocol is time consuming, requiring approximately 1 h per patient, and is difficult for a non-specialist to perform.

Thermography consists of observing and detecting the emitted irradiance from an object, in this case tissue. The method attempts to assess tissue perfusion based on the surface temperature of normal and suspicious tissue. It is of note that thermograhic methods applied to tissue probe only the first few microns (<100 microns) of the tissue, and room and patient temperature variations cause havoc on the measured values.

Magnetic Resonance Imaging can be applied to examine a variety of disorders, ranging from skin lesions to leg ulcers, by examining metabolism in vivo in a non-invasive manner. However, the time necessary to acquire an image, the total cost of a single unit and the limited mobility and portability make this method clinically impractical in this field of use.

Photoplethysmography is defined as the continuous acquisition of the intensity of light scattered from a given source by the tissues and collected by a photodetector. Photoplethysmography measures changes in blood volume by monitoring intensity changes in the observed signal that arise from the pulsatile change in blood volume in the blood vessels. Tissues that have a reflected light signal with a large pulsatile modulation are assumed to have a good arterial supply of blood. This technique has been primarily used to determine the sufficiency of arterial blood supply to the extremities, particularly the toes and fingers. The method measures the strength of the pulsatile modulation of the optical reflectance signal, which in turn is related to a change in blood volume. This measure is extrapolated as an indicator of blood supply to the tissue. The method does not report information related to oxygenation, a vital parameter in tissue health and viability and the technique is dependent on the tissue having a distinct pulsatile modulated blood volume which is typical only of highly vascularized tissue.

U.S. Pat. Nos. 4,223,680 and 4,281,645 both to Jöbsis describe a method and apparatus for in vivo monitoring metabolism in body organs using near infrared light. This is accomplished by measuring the absorption characteristics associated with the cellular metabolism of cytochrome aa₃. However, this apparatus uses a particular set of measuring and reference wavelengths to measure changes and trends in the metabolic activity of an internal body organ. Jöbsis also specified in both patents that the near infrared light must span a relatively long path (several centimeters) through bone, skin and tissue to the organ of interest for his invention to work.

U.S. Pat. Nos. 5,161,531 and 5,127,408 both to Parsons, et al. describe an invasive method and apparatus for in vivo monitoring of internal body organs such as heart, brain, liver and kidneys with the use of fiber optic probes and an elongated catheter. Specifically, the apparatus makes measurements pertaining to the oxygen availability and utilization in internal body organs and not cutaneous (skin) tissue. Likewise, U.S. Pat. No. 4,513,751 by Abe et al. describes an invasive method and apparatus that follows oxygen metabolism in an internal organ.

PCT Patent 9608201A by Vari and Maarek describe a non-invasive spectroscopic apparatus and method to assess burn injuries. The apparatus depicted specifically targets the use of selected wavelengths to assess the burn injury by evaluating the intensity of the fluorescence and tissue attenuation at these specific wavelengths. The device described therein does not acquire a multitude of discrete wavelengths comprising a spectroscopic response for a given wavelength range; rather, the aforementioned device looks at the intensity (or counts) and compares this to a database of normal tissue to assess the injury. In other words, the device lacks the ability to look at the attenuation as related to tissue absorption to delineate tissue viability. Burn injuries are classified according to the depth of the burn injury, no mention of the burn depth is disclosed by Vari and Maarek.

WO92/15008 to Rava et al teaches using laser light for diagnosis as well as treatment and/or removal of tissue. Specifically, the described device includes a laser catheter for removing plaques from a vessel wall as a method for treating atherosclerosis.

WO96/07889 to Vo-Dinh teaches a method of laser-induced synchronous luminescence for analyzing tumors and other tissues using dyes.

WO99/22640 teaches a device for the detection of various tissue states by observing various optical phenomena (emission and reflectance) using various illumination sources (UV, IR, far IR, and lasers). It is further stated that the device will use a database containing previous spectra for comparison purposes when determining tissue status. However, no clear outline of how this will be accomplished or a description of the device is provided. Furthermore, no indication of the processing methods or algorithms is provided, nor is any data shown. In addition, WO99/22640 does not consider the need to distinguish between surface and subsurface tissue absorptions or describe any steps for enhancing and analyzing data obtained from the spectra.

As discussed above, prompt and effective assessment of tissue following surgery or injury promotes a proper course of action, reduces the need of unnecessary medical attention, and aids in the restoration of the damaged tissue. Clearly, an apparatus that provides an early means of determining the status of tissues that are potentially threatened as a result of trauma, a chronic condition, disease state or a surgical procedure is required. The apparatus would preferably determine the status of tissue in a non-invasive, and non-subjective manner. The apparatus can also provide long term non-subjective re-assessment of tissue during the recovery process. This long term usage is essential in areas such as chronic wounds where the healing process can span several months or years. The apparatus can also be used at the time of surgery to determine the efficacy of a surgical procedure.

In view of this, Sowa et al (WO98/44839) describes a method of using near infrared spectroscopic imaging to assess tissue viability. Specifically, visible and near-infrared spectroscopy is used to analyze tissue hydration and oxygenation. The data are acquired simply, rapidly and non-invasively. Furthermore, the data from a single spectrum is sufficient, using the method described therein, to predict tissue viability, obviating the need to continuously monitor trends. The relative change and distribution of the levels of oxyhemoglobin (HbO₂), and deoxyhemoglobin (Hb) in tissue is examined and used to predict tissue viability. The near-IR and visible absorption spectra of Hb, HbO₂ and water are well understood and the differential absorption by these chromophores can be distinguished at certain characteristic wavelength regions (Eaton and Hofrichter, 1981, Meth Enz 76:175-261). However, there are several factors which must be taken into consideration and several limitations overcome when designing a device to carry out this method. Specifically, the light source must have sufficient light in the vis-near infrared range and the source must be stable. Corrections for curved surfaces and translational, rotational and scaling corrections for image registration must also be taken into account. Components capable of distinguishing between tissue surface and subsurface phenomena and detecting and differentiating between small signals must be designed. Furthermore, the device must be arranged to carry out a number of tasks, including, for example, tissue assessment at multiple points and at multiple depths, as well as two-dimensional imaging of an injured area.

### SUMMARY OF THE INVENTION

According to a first aspect of the invention, there is provided a device for single or multiple point spectroscopy for determining status of a tissue portion at the surface of the tissue comprising: a light source emitting energy in the wavelength region between 400-2500 nm; an illuminator delivering light from the light source to the tissue surface; a collector receiving remitted light from the tissue surface; a detector measuring wavelength data from the remitted light; an analyzer analyzing the data from the detector for measuring tissue viability; and a display unit displaying results from the analyzer.

The device may include a wavelength sensitive element for dispersing the collected remitted light into wavelength dependent components. As will be apparent to one knowledgeable in the art, this includes dispersive and non-dispersive elements such as gratings, prisms, acousto-optical tunable filters (AOTFs), liquid crystal tunable filters (LCTFs) and the like.

The device may include at least one optical path router for switching between single and multiple illuminators and collectors.

The optical path router may be connected to the detector for switching between a single collector at a single tissue site and multiple collectors detecting several tissue sites.

The optical path router may be connected to the light source for switching between a single illuminator illuminating one tissue site and multiple illuminators illuminating several tissue sites.

The optical path router may be connected to the detector for switching to obtain a sample of the illumination source.

The optical path router may be connected to the detector for switching to obtain a sample of the illumination source passing through a wavelength calibration standard.

An optical path router or shutter may be connected to the detector for switching to obtain a dark spectrum when no light is transmitted.

The optical path router or shutter may be connected to the illumination source for switching the transmission of source illumination off. This may be used to troubleshoot ambient light problems or probe/tissue contact problems.

The collector and the illuminator may be at a fixed distance relative one another for determining optical depth.

The detector may include a two dimensional detector.

According to a second aspect of the invention, there is provided a device for imaging spectroscopic analysis of a tissue portion comprising: a light source emitting energy in the wavelength region between 400-2500 nm; an illuminator delivering light to the tissue portion; a collector receiving remitted light from the tissue portion; a detector having a two dimensional sensor array for acquiring images at selected wavelengths from the remitted light; imaging devices detecting wavelength-dependent images from the detector; an analyzer processing the images into parameters for assessing tissue status; and a display unit for displaying the parameters.

The device may include an optical path router mounted to the collector for receiving remitted light from multiple sites within the tissue portion.

The device may include an optical path router mounted to the illuminate a single and multiple site within the tissue portion.

According to a third aspect of the invention, there is provided a device for multiple point spectroscopic analysis of a tissue portion comprising: a light source emitting energy in the wavelength region between 400-2500 nm; a probe head having mounted thereon: an illuminator illuminating the tissue portion; collectors gathering remitted light from the tissue portion, each of said collectors being mounted on the probe head at a position distal to the illuminator and one another for acquiring spectral information at a given tissue depth; a plurality of detectors dispersing the remitted light gathered by the collectors into wavelength dependent components, each detector being linked to a respective one of the collectors; an analyzer processing the wavelength dependent components into parameters for assessing tissue status; and a display unit for showing the parameters.

The light source may be modulated.

The detectors may detect the remitted light in the time or frequency domain as the light source modulates.

### BRIEF DESCRIPTION OF THE DRAWINGS

FIG. 1 shows the spectrum of the Hb, HbO₂, water, and the difference spectrum of oxidized minus reduced cytochrome aa₃, to describe the chromophores or parameters one can obtain with the apparatus.

FIG. 2 shows a diagram of depth spectroscopy setup a) general instrument diagram b) sampling depth with multi-point spectroscopy.

FIG. 3 is a typical response for the depth spectroscopy apparatus a) response of an optical standard b) dark noise response c) reflectance response from the surface of normal skin.

FIG. 4 shows the basic apparatus concepts.

FIG. 5 shows embodiments of the apparatus wherein a modulated light source is utilized.

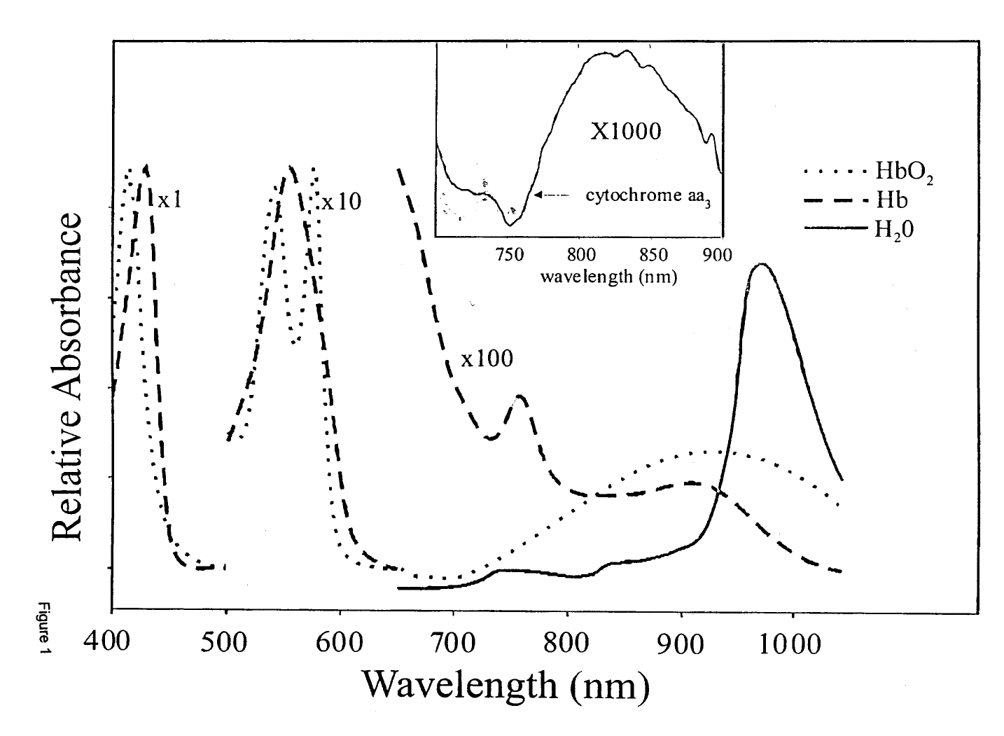

### DESCRIPTION OF THE PREFERRED EMBODIMENTS

Unless defined otherwise, all technical and scientific terms used herein have the same meaning as commonly understood by one of ordinary skill in the art to which the invention belongs. Although any methods and materials similar or equivalent to those described herein can be used in the practice or testing of the present invention, the preferred methods and materials are now described. All publications mentioned hereunder are incorporated herein by reference.

Definitions

As used herein, tissue viability refers to the state of the tissue with regards to whether or not the tissue will survive if no further action is taken.

As used herein, tissue health refers to the state of the tissue with regards to proper tissue perfusion, oxygenation saturation, oxygen consumption, and water content.

As used herein, tissue status refers to the current state of the tissue with respect to the current status of the tissue chromophores, health and viability.

As used herein, abnormal or compromised tissue refers to tissue in some sort of flux or perturbation from its original status prior to injury, disease, the onset of a condition, or surgical procedure.

As used herein, thermal injury refers to an injury caused by either extreme cold or heat which alters or damages the tissue, chemical or electrical burn which alters or damages the tissue, or chemical or electrical trauma which alters or damages the tissue.

As used herein, systemic refers to the entire system or whole body, for instance systemic oxygenation refers to the oxygenation status of the blood circulating through-out the body.

As used herein, tissue oxygenation refers to oxygenated hemoglobin ratio of blood contained in the arteries, veins and capililary compartments of the sampled tissue volume.

As used herein, oxygenation refers to the ratio of hemoglobin carrying oxygen to the amount of hemoglobin that is oxygen depleted. Tissue oxygenation refers to the ratio of oxygenated to total hemoglobin in the blood contained in the arteries, veins and capillary compartments of the sampled tissue volume.

As used herein, blood volume or total hemoglobin refers to a combined measure of oxygenated and deoxygenated hemoglobin, which can be used as an indicator of tissue perfusion.

As used herein, hydration refers to amount of fluid present both lack of or accumulation resulting in a significant decrease or increase in tissue volume.

As used herein, chronic wound refers to a medical state wherein there is a persistent injury and the normal healing process is impaired.

As used herein, contact refers to a state of interaction or touching of the tissue with the apparatus.

As used herein, non-contact refers to a state of immediate proximity without touching or disturbing the tissue.

As used herein, non-invasive refers to a procedure whereby the tissue is unaltered from it's present state and non-intrusive As used herein, minimally invasive refers to a procedure whereby the tissue is minimally and unnoticeably adjusted to permit the apparatus to obtain meaningful measurements.

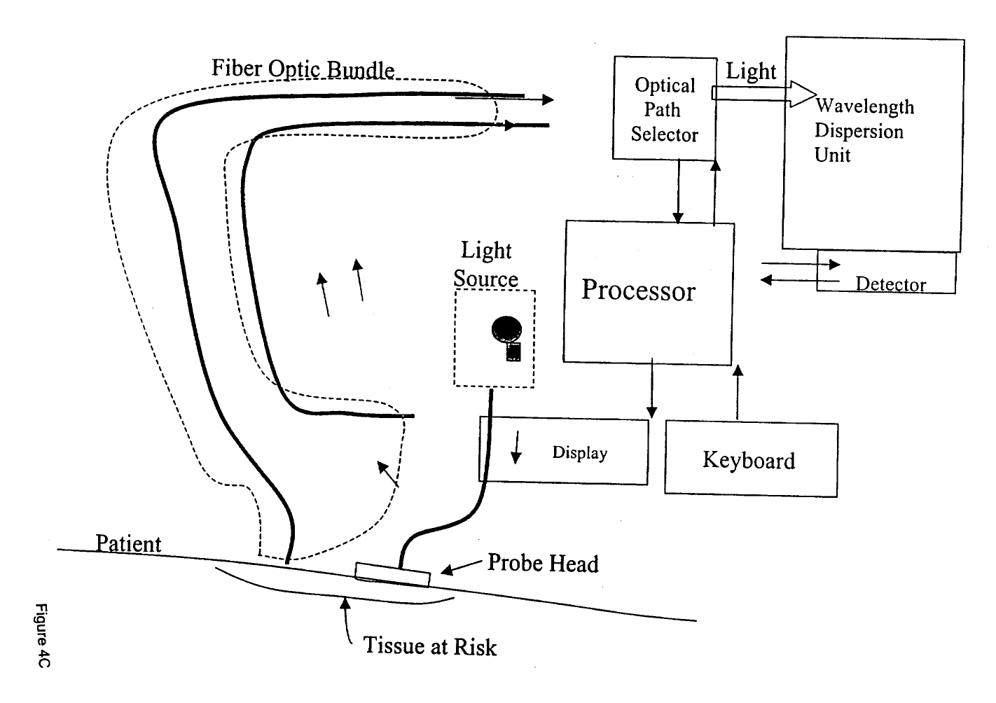

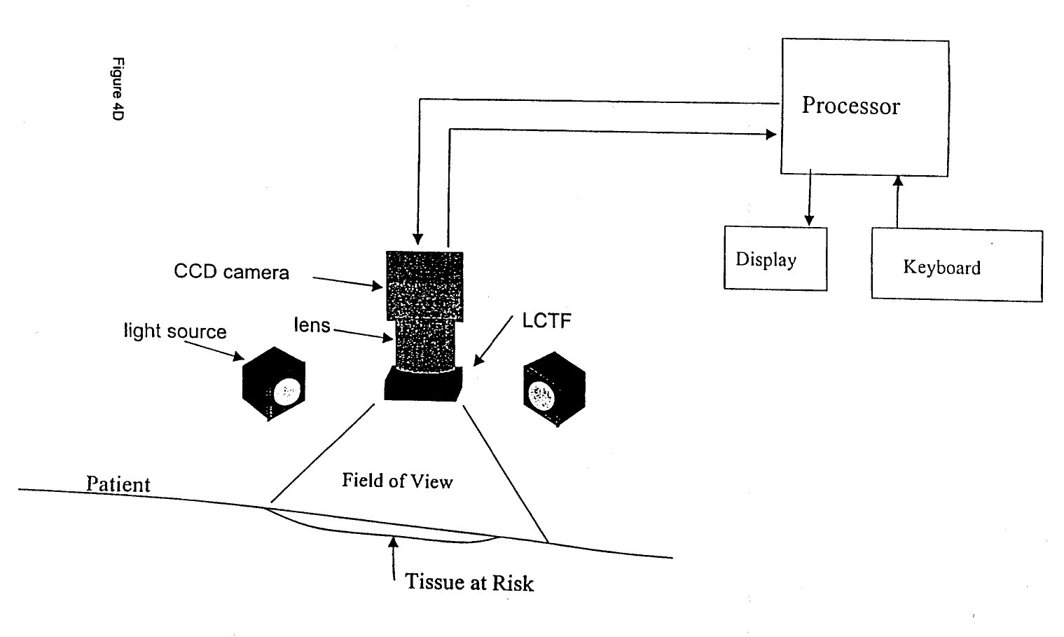

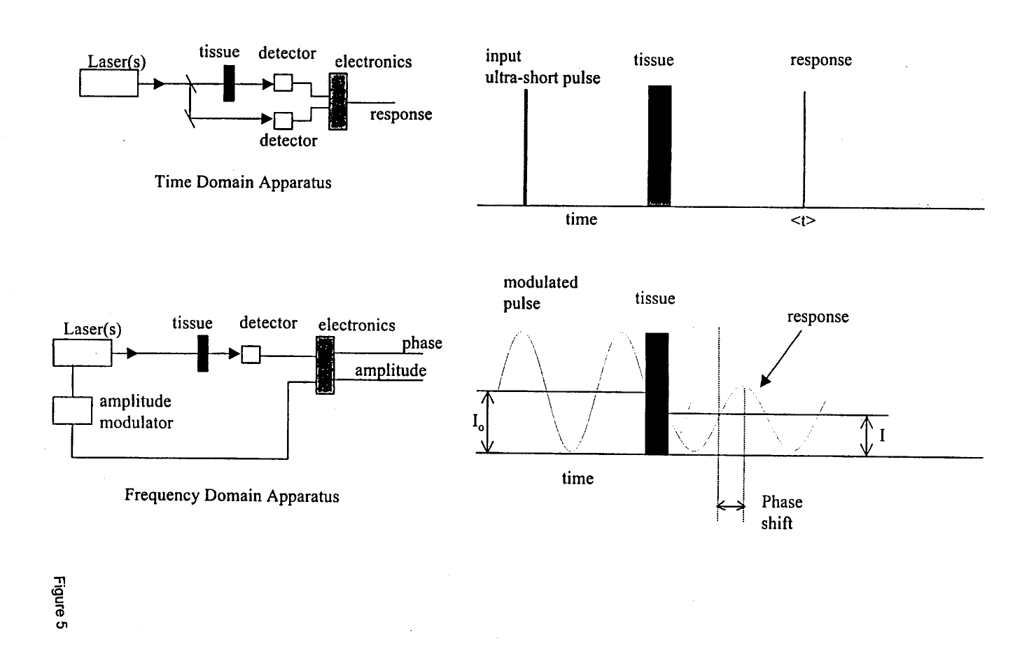

Described herein is a device for use in assessing tissue viability, status and health, as shown in FIG. 4. The device comprises a light source, an illuminator/collector, a detector, an analyzer and a display unit. The apparatus provides information on tissue viability in a non-invasion manner utilizing visible and near infrared absorption reflection spectroscopy. The tissue viability is based on measures of the chromophores deoxyhemoglobin (Hb), oxyhemoglobin (HbO₂), water (H₂O) and others that may be present in the tissue, as taught in WO98/44839, which is incorporated herein by reference.

FIG. 4 describes the general concept of the apparatus broken down into the various components: Light source, illuminator/collector, detector, analyser, and display.

The light source provides light illumination to the tissue. The light source may comprise, for example, a light source emitting energy in the visible and infrared range encompassing the wavelength region between 400 and 2500 nm.

The illuminator delivers the light to the tissue and the collector receives information from the surface and from this gathers the spectroscopic information from the tissue. The collector may, for example, collect the wavelength dependent components of the remitted light, collect remitted light at selected wavelengths for developing images or collect wavelength components of remitted light from the tissue surface at several radial positions away from the illumination source, as described below.

The collector may, for example, collect the wavelength dependent components of the light that are remitted from the tissue. Selected discrete wavelengths of the remitted light can be collected for developing multi-spectral images or responses. A continuum of wavelengths can also be collected to provide hyperspectral and spectroscopic image data or spectra. The remitted light can be collected from one or more radial positions away from the illumination source, as described below.

The detector unit disperses the light into the various wavelength components and tracks/records the intensity of the various wavelength components from the collector. The detector unit may, for example, detect reflected light energy from one or more tissue sites or from areas of tissue. The detector unit may also detect a portion of the light source for determining the system or instrument response. The detector unit may be an optical detector, a fiber optic detector, or a lens based optical system, as described below.

The analyzer receives the spectroscopic information from the detector unit and analyzes this data using computation formulas to provide a meaningful measure of tissue viability. The analyzer may, for example, process the wavelength dependent spectroscopic profiles, or wavelength-dependent images into sets of parameters used to assess the status, viability or health of the tissue in near real time.

The display unit displays the information from the analyzer on either a visual display or as a printout. This information may be displayed in near real-time.

An example of the field of use of the apparatus by a medical practitioner is in the assessment of the condition of tissues in various conditions of health ranging from tissue which is healthy through tissues which are at risk of becoming necrotic. A comparison of healthy and near necrotic tissue shows a stark contrast in their near infrared spectra, and the oxygenation and total blood volume and hydration parameters derived from the near infrared spectra.

In one embodiment of the invention, there is provided an apparatus for single or multiple point spectroscopy for determining the status of tissue. In this embodiment, the light source produces light to illuminate the tissue, the illuminator/collector delivers and collects the remitted light from the tissue surface as well as a means of collecting a portion of the light source to determine the system response. The detector unit is a spectroscopic optical means, that measures the wavelength dependent components of the remitted light from the tissue. The wavelength dependent components are used to obtain parameters used in the determination of tissue status. The analyzer processes the wavelength dependent spectroscopic profiles into parameters used to assess the status, viability, or health of the tissue in near real time, the results of which are shown on the display unit. In this embodiment, the device also includes a wavelength selector that disperses the remitted and reference light into its wavelength dependent components. The wavelength dependent components are dependent on the state of the light source, the apparatus and status of the tissue.

Specifically, in this embodiment, the device comprises a light source emitting energy in the visible and infrared range encompassing the wavelength region between 400 and 2500 nm, an optical means of illuminating the tissue, and an optical means of collecting the light that is reflected, transmitted or scattered from the tissue site. In this embodiment there is a means whereby the distance separating the delivery and collection optics can vary by some known distance on the surface of the tissue. In this embodiment there is a spectroscopic system designed for dispersing the electromagnetic spectrum into its respective wavelength components and converting this information into parameters that are related to the status of tissue. In some embodiments, this requires digitization of the spectrum but in other embodiments, may include optical computations as well.

The spectroscopic system must meet the criteria of producing a spectrum of sufficient wavelength resolution, of sufficient signal to noise ratio and a spectrum containing sufficient region(s) of wavelength information.

In this embodiment, the device also includes an optical path router (OPR) capable of switching between multiple optical inputs and outputs either using a single or plurality of these. The OPR can function in a reversible manner as well, in that it may have a single input and multiple output switched paths. This reversibility of the OPR allows the device to be situated either before the detector unit to select from multiple inputs, or after the light source to direct the light to several output targets. The OPR's may exist in the various permutations in the apparatus to provide both selection of the illumination target and/or selection of the reception site. In some embodiments, there may be provided a device without an OPR. It is of note that the OPR can be capable of transmitting a point source, one dimensional linear source, or two dimensional imaging source. Also, more than one OPR many be chained together to increase permutations of functionality.

In this embodiment, there is provided a controller/analyzer unit which controls the various sub-assemblies in the device, controlling the detector and obtaining spectra from it, controlling the OPR('s) if the apparatus contains one, monitoring the illumination source, and the analyzer processes the obtained spectra into medical meaningful data.

In this embodiment, the analyzer calculates medical diagnostic parameter(s) on tissue viability from the spectroscopic information acquired by the detector, for example, data relating to hydration, hemoglobin, oxygen bound hemoglobin, oxygen saturation as well as total hemoglobin.

The display unit allows for visualization of the calculated information.

In some embodiments, the device may include an input device such as a keyboard to allow for user interaction with the apparatus.

As a result of this arrangement, the apparatus provides information on tissue viability in a non-invasion manner utilizing visible and near infrared absorption reflection spectroscopy. The tissue viability is based on measures of the chromophores deoxyhemoglobin (Hb), oxyhemoglobin (HbO₂), water (H₂O) and others that may be present in the tissue.

As discussed above, the apparatus operates by delivering and receiving visible and near infrared light in the range 400 to 2500 nm to the tissue site of interest. Light in this region of the electromagnetic spectrum is ideal for tissue viability and health assessment due to the attributes of weak absorption by the tissue and high forward directed scattering by tissue constituents. This combination of weak absorption and high scattering permits the light to penetrate a substantial distance within tissue. Since tissue is a highly scattering medium, there is strong inverse correlation between the amplitude of the returned signal and depth of tissue sampled. This decrease in reflected signal strength and the detection limit of the detector unit limits the maximum depth of tissue that may be probed. The spacing between the fiber optics of the illuminator and collector determines the mean optical depth that light can penetrate into the tissue. The reflected light provides input for the detector unit, which may contain a grating spectrometer known in the art, which disperses the light of the electro-magnetic spectrum into its wavelength components. This dispersed spectrum is then directed on a linear array detector, which converts the light into an electrical signal. This electrical signal is then digitized and transferred to the analyzer creating a digital spectrum of the tissue. The spectrum is then processed using computational algorithms described in WO98/44839 and the results are displayed. A keyboard may also be provided to allow for user interaction with the apparatus.

It is worth noting that the optical path router (OPR) is capable of switching between multiple optical inputs and directing either a single or plurality of these to an output port that can be positioned at the entrance of the detector unit. The selection of multiple inputs allows for the selection of reflected light from one or more tissue sites, light directly from the illumination source, or no input light to measure the dark or null response of the apparatus. These spectra allow for the calculation of an optical density (OD) spectrum given by the relationship, OD=log₁₀ ((illumination spectrum-dark spectrum)/(tissue spectrum-dark spectrum)). The ratioed responses provide information related to the attenuation of light at the particular wavelength, which is ultimately related to the absorption of the tissue chromophore at that particular wavelength. Furthermore, the dark response of the system may be used to further correct for the instrument response, thereby improving the quality of the tissue spectrum

The apparatus may also have multiple tissue probes with the OPR acting as a multiplexer allowing the apparatus to scan several tissue sites. These sites may include both tissue sites of suspicious health and sites of healthy tissue. In addition, the inputs may include: light from the source passing through a wavelength calibration standard, light from a single or multiple discrete source(s) such as a laser diode for calibration purposes, multiple tissue sites which may include reference sites, a second apparatus or another patient. Multiple or combinational OPR's may exist in the apparatus to provide both selection of illumination target and selection of the reception site.

The OPR can be capable of transmitting either a point source, a one-dimensional source, or a two-dimensional source. In a point source configuration, the organization and orientation of the light transmission is not important and a simple detector unit utilizing either a linear detector or a column binned two-dimensional detector or area detector. The one-dimensional configuration allows for multiple channel throughputs into a detector unit equipped with an area detector. This allows for several channels to be recorded simultaneously. The optical isolation of the channels is retained through the OPR and detector unit so that each channel can be distinguished when imaged on to the detector. It is of note that such a system with an area detector could be used without an OPR and still sample several sources including tissue sites, source reference, backgrounds and others using other forms of channel multiplexing. An area detector may require a shutter during readout or for background spectrum collection. In place of the area detector and shutter, a frame transfer detector may also be used. It is of note that in some embodiments, a device may exist with a combination of OPR(s) and/or multiple detector units. The OPR may also transmit two dimensional source imagery which may be used in imaging systems, discussed below. A system may also exist without an OPR or channel multiplexer.

The OPR differs between the conventional fiber optic switches used in fiber optic communication networks in that the OPR transmits broadband light, usually using multimode fiber optic cable. The OPR is usually involved in the transmission and coupling of bundles containing multiple fiber optics. The concept of the OPR includes: reproducible switching between inputs or outputs, high coupling efficiency and minimal throughput loses of the transmitted signal(s), minimization of channel cross-talk or interference and minimization of light leakage when OPR is set to transmit no signal.

It is of note that when a system is constructed without an OPR, several challenges are presented. For example, the light source is either monitored through some other mechanism or it is assumed to be stable during the tissue assessment. In addition, the collection of a reference spectra is done manually, which opens up the possibility of user error in the collection of the reference. Furthermore, the lack of an OPR limits the device to processing difference values for monitored tissue parameters and the system to a single probe if the detector unit is equipped with a single element or one-dimensional detector. These drawbacks may be managed through the use of multiple fibers and an area detector assuming the response on the detector is known and stable.

Thus, a system that incorporates an OPR is considerably more flexible. Firstly, the OPR allows for collection of a reference spectrum at any time point. This is important not only in the computation of OD spectra but also as self-diagnostic or system troubleshooting algorithms. In a similar fashion, the incorporation of a wavelength calibration standard as an input channel can also be used for apparatus diagnostic purposes and performance tracking. As a result of this arrangement, the system can check the dark spectrum for system performance in an apparatus diagnostic methodology and use this information to self-correct system performance. The OPR combined with a single element or one-dimensional detector enables the usage of multiple sampling sites or depths. When combined with a two-dimensional detector and the appropriate illuminator/collector the same system may either collect multiple optical channels with a two-dimensional detector or capture a single weak spectrum using the detector in a binned format. When an OPR is coupled to a light source, the apparatus can then sample multiple sites while maintaining maximum light delivery to the current site of queue and therefore maximum signal quality.

The sampling probe contained in the illuminator/collector can be as simple as separate delivery and reception fiber optics at a known or controlled separation. Usually, the delivery and reception fibers will be integrated into a single probe head with a set spacing. The probe may also contain a pressure and/or shear stress sensor linked back to the apparatus to indicate the force being applied to the tissue beneath the probe. Pressure or shear stress forces involve movement of interstitial fluids and blood of the tissue, which affect the concentration of chromophores being detected from the tissue beneath the probe.

The sampling probe may contain a temperature sensor to account for temperature variation of the tissue. The optical and spectroscopic properties of tissue are temperature dependent. Tissue can be thought of consisting of a medium composed of microscopic constituents of varying refractive index. Since the refractive index and density of a medium are strongly temperature dependent, changes in the observed response are expected. With an increase or decrease in temperature, the density and optical properties of the medium are altered, thus changing the response from the apparatus. Temperature also plays a major role with respect to oxygen diffusion in tissue and can provide erroneous results if these effects are not accounted for in the analysis. Alternatively, temperature may be calculated from the tissue spectrum. Water and its temperature dependent spectral variation has been a field of interest in biomedical tissue spectroscopy. The extinction coefficients of water are extremely temperature dependent, such that a temperature variation will cause a spectral shift in the spectrum. A temperature increase will cause the water spectrum to shift to shorter wavelengths. Therefore, the temperature of the tissue can be monitored using the response in the water spectral region

The illuminator/collector probe may also contain a reference fiber set for the correction of fiber absorbances and loses caused by fiber core material, fiber impurities, fiber cladding, mechanical stress or other sources. This reference fiber set may contain a loop in the probe head or may contain a reference standard to reflect off of. The reference fiber set may consist of one or more fibers with input from the illumination source and output back to the apparatus. The purpose of the reference fiber is to provide a reference spectrum of the illumination source and a spectrum of the result of the illumination light's interaction with the fiber. The spectrum collected from this reference fiber when used in the computation of optical density as stated above should account for the fiber and system effects in the resulting tissue sample spectrum. Other methods of collecting a reference spectrum, disparate from the probe reference method, may include a separate fiber optic within the device of similar fiber characteristics with input from the source and output to spectroscopic section of the device. A manual reference can also be acquired by manually placing the probe on a reflectance standard. Another configuration may involve monitoring the source at one or more discrete wavelengths and using this information to account for source stability, although this method does not account for effects that the fiber optic may have on the attenuation of the signal. If changes in tissue condition or differences between tissue sites are being monitored, then a sample from a previous measurement or other physical site may be used to ratio out the system's effect on the tissue sample site of interest.

The probe may also contain a tag, which is capable of identifying the probe to the main device unit. This tag may contain information such as probe model number, a unique serial number, and information about the probe or configuration information for the device. The tag may also contain write-able memory, which may contain information on probe usage or other updateable information.

With the flexibility provided by the OPR, the apparatus may also be composed of more than one probe head to sample more than one tissue site. The sites may be composed of various permutations involving tissue of suspicious viability and healthy tissue or other configurations to provide the medical practitioner with the data stream they desire. An application of this would be to compare and contrast the suspicious tissue sites against a known healthy tissue site. An alternate probe configuration may consist of multiple illuminator and collector probes separated at various distance from one another. This permits sampling of multiple depths into the tissue and the investigation of the various layers of tissue beneath that which is visible. An application of this would be to profile the depth of thermal damaged tissue to differentiate intermediate partial thickness burns from deep partial thickness burns, as described below. The probe arrangement may involve a combination of discrete probe sites and depth profiling sites.

An additional embodiment of the apparatus may include a high rate scanning mode. This mode is intended to distinguish and identify the extent of the abnormal tissue across the surface. The high rate scan mode consists of moving the probe across the surface of the suspicious and normal tissue and collecting, processing and displaying the acquired information. In this mode, the boundaries between healthy and abnormal tissue can be quickly identified. This high rate scan mode may be accomplished manually, mechanically, optically, or by some other means. In order to enable this mode of operation, the detector exposure time per spectrum must be shortened to the point that motion artifacts are minimized in the spectrum. To accomplish the short exposure time and maintain a reasonable signal level a detector of higher efficiency is required and a detector cooler may also benefit the system by reducing dark noise. In addition, processing algorithms can detect artifacts from excessive motion and disregard those spectrum and signal the system that the motion is causing artifacts. The algorithms may be altered to deal with increased data flow, changed spectrum characteristics and other differences from standard operation. The system may also incorporate a trigging device for the user to capture data at their discretion.

A major benefit of using broadband spectroscopy is that the scattering contribution to the light attenuation by tissue may be determined and corrected over narrow regions of the spectrum. Scatter correction is not available to discrete wavelength systems simply because they do not provide sufficient data as function of wavelength to afford a reliable correction. The ability to qualify the scattering is what gives this apparatus the ability to give reproducible results even after repeated probe removals and reattachments, and to account for patient and probe movement. Both of these factors are a major failing of prior art devices in this area.

In addition to the techniques discussed in WO98/44839, the following technique may also be used to extract information about the tissue from the measured (recorded) spectra. Herein, hydration values are obtained from the 980 nm water band region and hemoglobin values are obtained from the 700-840 nm region. The relative concentrration of these chromophores are derived from the specified regions of the spectrum by a least squares fit of the chromophore extinction coefficients to the measured spectrum. In addition, possible water effects can be removed from the results by using prior calculated hydration values, and then carrying out a least square fit of the oxy-hemoglobin, deoxy-hemoglobin and other optical effects. The chromophore concentrations derived form the tissue spectrum may now be used to calculate the ratio of oxygenated to deoxygenated hemoglobin, the combined oxygenated and deoxygenated hemoglobin or total hemoglobin and the ratio of oxygenated hemoglobin to total hemoglobin.

In a further embodiment, the device is arranged for imaging spectroscopy to determine the status of tissue. In this embodiment, the light source produces light to illuminate the tissue used to measure the absorption or reflectance from the tissue to provide a tissue spectrum. The collector receives the remitted light from the tissue surface using a lens-based optical system. The detector unit selects the wavelengths using a non-disperse device to collect remitted light from the tissue in an imaging fashion using a two-dimensional sensor array to acquire images at selected wavelengths. The analyzer processes the wavelength dependent images into parameters used to assess the status, viability, or health of the tissue in near real-time on a display.

As discussed above, the point spectroscopic apparatus provides an excellent means of evaluating tissue viability, status and health when the area being assessed is clearly known or only a select few areas are necessary. However, in some cases, the dividing line between health and necrosis or disease is not easily distinguishable i.e. there are varying degrees of poor perfusion or damage across the surface of the tissue. Several apparatus can be used to obtain total tissue surface health information. One approach involves sampling multiple locations on the tissue surface. A second approach comprises the use of multiple optical fibers at multiple locations across the tissue site of interest. An alternative to both these techniques employs an apparatus capable of obtaining spectroscopic information all at once in an imaging fashion. Such a method has the advantage of acquiring spatial tissue health information instantaneously.

Imaging devices are the most well known and used of the photonic detectors. In general, imaging devices/detectors convert light or photons into an electrical charge that is collected and stored in a metal oxide semiconductor. The accumulated charge or response is a linear function of the incident and exposure time to the light energy. The response from an imaging device represents the reflected spatial light intensity of a given object. In conventional imaging devices, the detected response observed is the accumulated intensity for a broad range of wavelengths. Therefore, an imaging device for in vivo tissue applications is of little use without some means of wavelength selection. Several methods can be applied to coupling a wavelength selection device to an imaging detector to provide spectroscopic spatial information to assess tissue viability, status or health.

An extension of the point apparatus is a system capable of providing spatial information on tissue viability in a non-invasive and non-contact manner using visible and near infrared reflectance/absorption spectroscopy. This can be accomplished using a spectroscopic imaging apparatus utilizing: an illumination source(s), a two dimensional detector and a wavelength selection system capable of passing two-dimensions of spatial information. One variation of the imaging apparatus is to use an imaging spectrometer and to allow a single column of a multicolumn image into the imaging spectrometer in a stepped fashion until all columns in the image are collected. Therefore, the image acquired consists of one spatial and one wavelength dimension. Another possibility would be the use of a point spectrometer in which the image is generated by scanning the input over the surface area. The result in all these methods is a three-dimensional cube of data consisting of x and y dimensions of spatial information and a z dimension containing wavelength information. The formats may include both micro and macro imaging, as well as varying degrees of image pixel density from those tight enough to create a visually recognizable image to someone trained in the art to a more sparsely spaced grid approaching that of a random discrete point map. In some formats the apparatus may take the form of a contacting probe system while still maintaining non-invasiveness.

The apparatus utilizes: an illumination source(s), a wavelength selection unit, a optics system to form an image, a two dimensional detector, an analyzer/controller, a display system, and a keyboard for user interaction. The device may also contain: a point spectrometer and OPR to monitor the source(s), a reference system to obtain information on detection system characteristics, a system to model contours of imaged surface such as laser scanner or stereoscopic images, a system to over-lay images captured from different surface and camera viewpoint configurations and possible different time points, a system to calculate relevant medical diagnostic images of tissue viability and a system to account for uneven illumination of scene due to shadowing or contour effects, image registration to correct for translational, rotational, and scaling uncertainties and the use of polarizers to distinguish between surface and sub surface phenomena.

An application of the imaging spectroscopy is in the field of tracking and measuring the status of chronic wounds under treatment. One of the methods of treatment for chronic wounds is hyperbaric oxygen therapy. The increase in oxygenation results in a contrast enhancement for spectroscopic imaging. The contrast is provided by increased oxygen inhalation by the patient. The increased contrast allows for increased differentiation of healthy and tissue which is at risk in both point and imaging applications.

In another embodiment, there is provided an apparatus for multiple point spectroscopy to determine the status of tissue comprising: a source for producing light to illuminate the tissue used to measure the absorption or reflectance from the tissue to provide a spectrum; an optical means (fiber optics) of illuminating the tissue using a light source; an optical means (fiber optic) to collect the remitted light from the tissue surface at several radial positions away from the illumination source as a method to acquire spectral information at various depths into the tissue; a device to disperse the remitted light collected at several positions into its wavelength dependent components which provides spectral information on the status of the tissue at various depths into the tissue; a device to detect the wavelength dependent components of the remitted light collected at several positions away from the source to obtain parameters used in the determination of tissue status; and a computational method to process the wavelength dependent spectroscopic profiles into parameters used to assess the status, viability, or health of the tissue in near real-time on a display.

The determination of tissue viability, status, and health following reconstructive surgery and/or a tissue-altering insult relies on the ability to accurately assess the tissue below its superficial layer. Such alterations modify the physical and optical properties of the tissue from the surface to deep within the tissue. As discussed above, the spectroscopic properties of the tissue can be acquired using either point spectroscopy or spectroscopic imaging. However, these methods are limited to sampling very shallow depths into the tissue therefore probing a thin portion of the tissue. In many cases however, the tissue assessment relies on the determination of the extent of viable tissue below the surface. Depth dependent tissue assessment can be accomplished by acquiring spectroscopic tissue responses at various depths into the tissue.

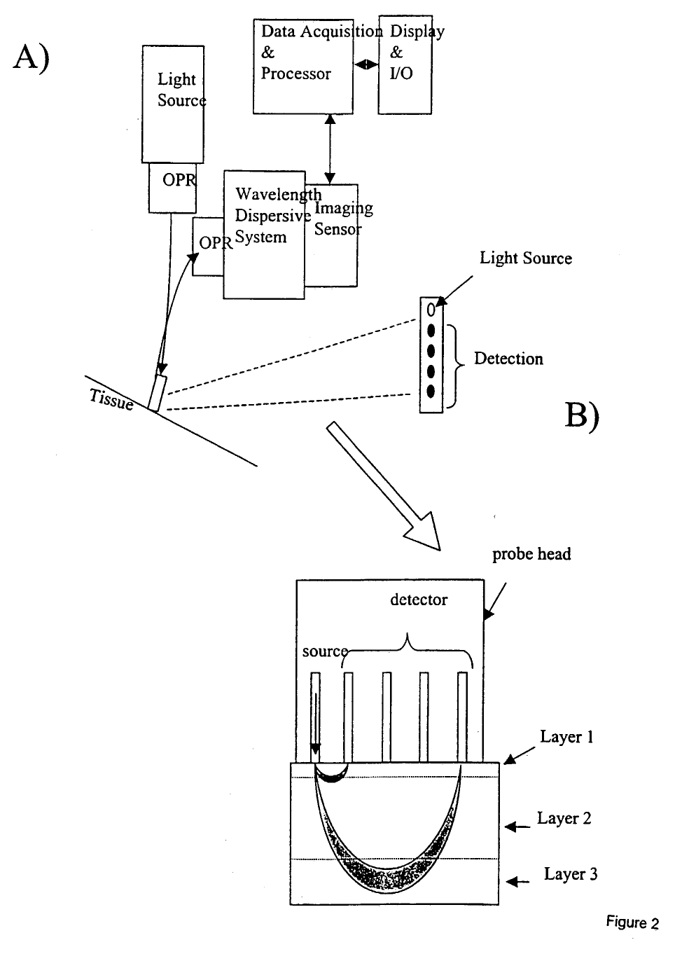

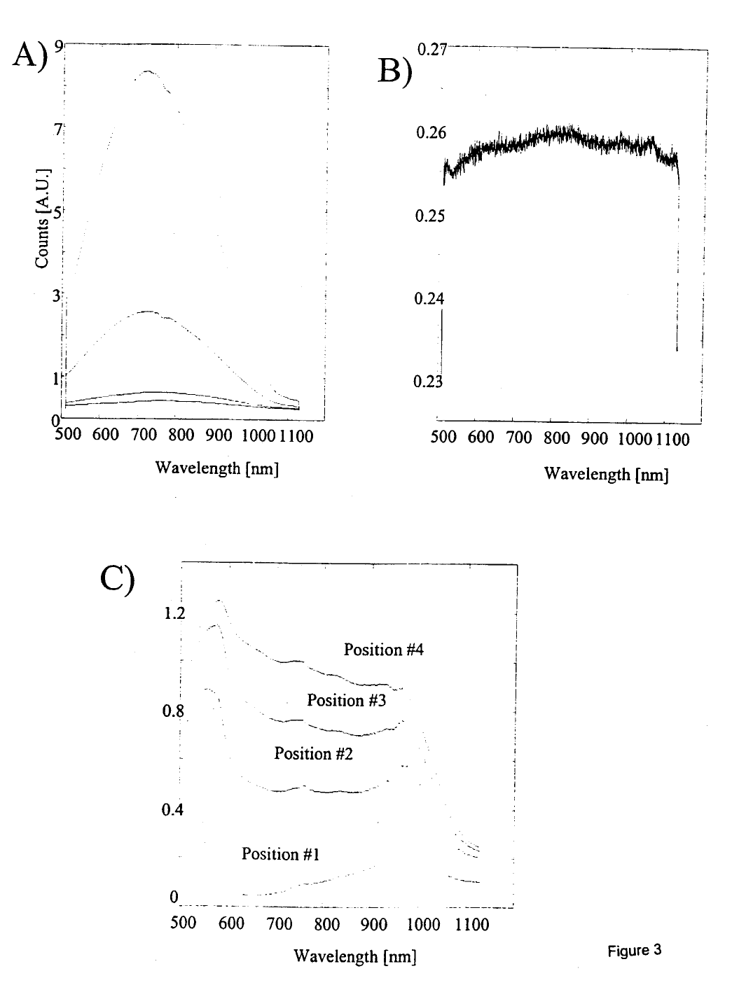

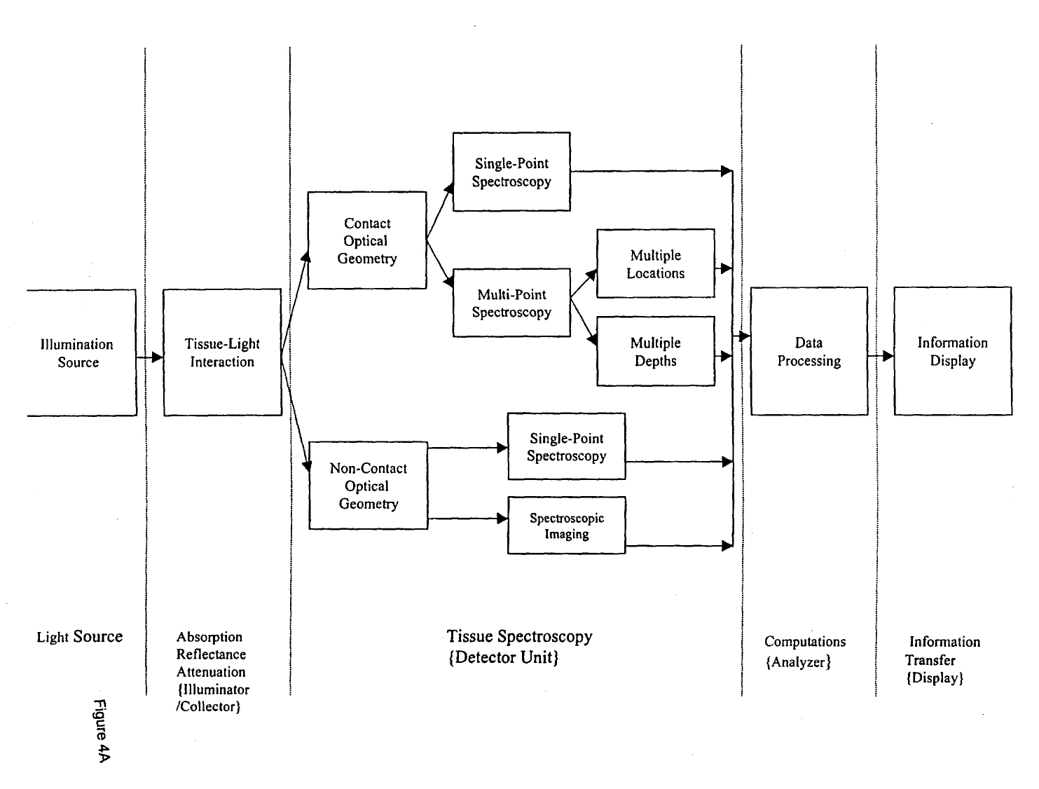

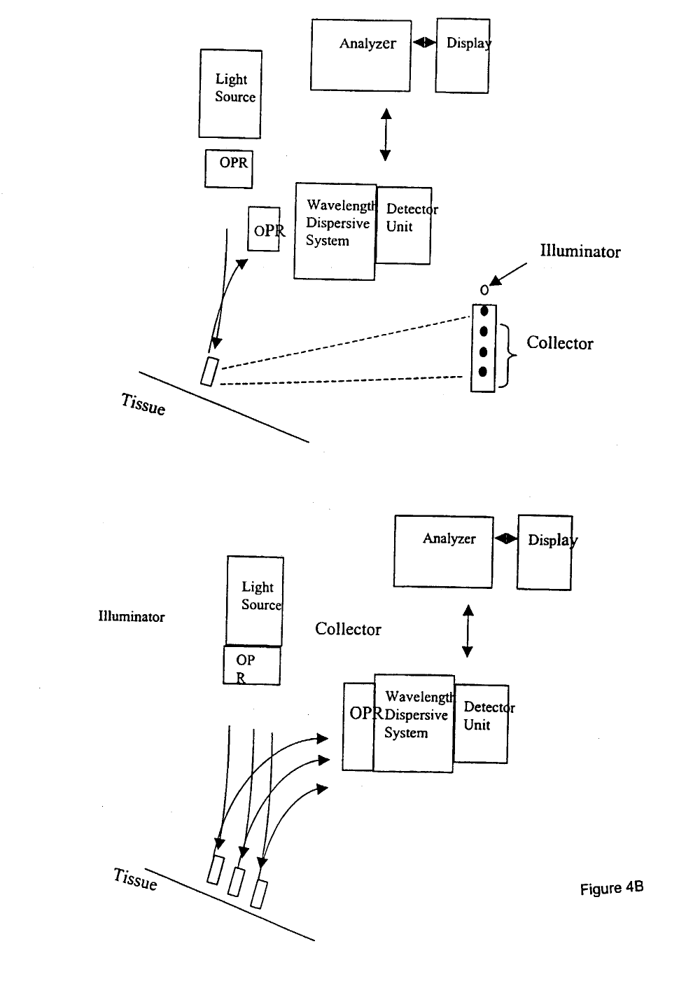

The transport of light through tissues is governed by the absorption of light by the tissue chromophores as well as the light-scattering interactions in the medium. Scattering of light occurs as a direct result of the interaction of light with random variations in the refractive index or small particles in the medium, resulting in a dispersion of the light in all directions. In the reflectance geometry of FIG. 2, a small fraction of the light penetrates into the tissue and is remitted out back to the surface. This remitted or diffusely reflected light collected by the detector has been attenuated as a result of the scattering as well as through absorption by the chromophores in the tissue. A measure of the reflected light provides spectral information on the scattering by the tissue and absorption by the tissue chromophores. FIG. 2 also depicts the detected light path through skin at various source-detector separation distances. When light enters a scattering media such as tissue, the light is preferentially scattered in a forward direction. In order for light to reach a detector a set distance away from the source, the light must traverse a path through the media, denoted by the shaded region. As the source-detector separation increases, the path increases and the depth sampled into the medium also increases. These paths have been described by a number of authors using both Monte Carlo simulations and single photon time correlated spectroscopic techniques. Essentially, collecting spectroscopic absorption spectra at various source-detector separations provides information on the tissue alteration or status at several depths into the tissue.

As shown in FIG. 4, the present apparatus permits the evaluation and assessment of tissue health, status and viability deep within the tissue. The apparatus employs visible and infrared light to monitor and evaluate the condition of the tissue based on the tissue optical changes encountered. Proper evaluation of the tissue following a surgical procedure or an insult to the tissue will allow for the correct course of action to be taken. This may result in one of two basic options: 1) to perform a surgical procedure to correct the problem; or 2) to allow the tissue to heal without surgical intervention. This apparatus is directed towards an unbiased non-invasive method to assess deep tissue viability.

The device shown in FIG. 4 includes a visible-infrared light source, a detector unit consisting of a wavelength dispersive instrument or module, and an area array detector or sensor, an analyzer consisting of a data acquisition and processor, and several optical fibers for the illuminator and collector. The detector for this application may be more sensitive since the signal levels at the deeper tissue levels will be lower. It is of note that in some embodiments, the detector may require cooling to decrease electrical noise levels to maintain good spectral signal to noise ratios.

In another embodiment, there is provided an apparatus for multiple point spectroscopy to determine the status of tissue in a given area comprising: a source for producing light to illuminate the tissue used to measure the absorption or reflectance from the tissue to provide an absorption spectrum; an optical means (fiber optics) of illuminating the tissue using a pulsed or modulated light source; an optical means (fiber optic) to collect the remitted light from the tissue surface at several radial positions away from the illumination source as a method to acquire spectral information at various depths into the tissue; a device to detect the remitted light collected at several positions away from the source to obtain parameters used in the determination of tissue status; a means of detecting the remitted light in the time or frequency domain as related to the illumination source to obtain tissue parameters associated with health or injury; and a means to display the processed time or frequency response to assess the status, viability, or health of the tissue in near real-time.

In general, as light enters into tissue two processes occur, absorption and scattering of the light. When tissue is illuminated with near infrared light, some of the light is absorbed by the tissue chromophores while a large portion of the light is diffusely scattered. Scattering of light occurs as a direct result of the interaction of light with random variations in the refractive index or small particles in the medium, resulting in a dispersion of the light in all directions. The observed or detected light is related to the concentration of scattering centers. Scattering alters the straight-line direction of the path that light propagates through tissue. Thus scattering results in an increase in the path-length that light travels through tissue relative to the straight-line path that light travels in a non-scattering medium. The increased path traveled by the light results in a greater attenuation of the light intensity due to an increased chance of light absorption by a chromophore compared to a non-scattering medium with comparable chromophore concentration. As a whole, the reflected light observed from the tissue is a function of the absorption by the chromophores and scattering from the constituents. Assessing tissue viability, health and status often requires the scattering contribution of the detected light to be distinguished or separated from the absorption contribution prior to the analysis and display processes.

The common approach to scatter correction in spectroscopy has been to use the entire spectrum or spectra in such routines as internal standards, second derivative data processing, and multiplicative signal correction to decrease the variability resulting from scattering. An alternative approach to scatter correction in tissue uses either photon time or frequency resolved techniques to obtain information on the mean paths light travels through tissue. Photon time-of-flight methods, a time resolved technique, use ultra-short pulses of illumination in which the light is diffusely scattered in the tissue. Semiconductor, dye, or solid state lasers produce the ultra-short pulses at discrete wavelengths in time-of-flight instruments. Photon paths through tissue can be obtained using these ultra short laser pulses and electronics to digitize and collect the response for a single photon event. Using a large number of photons, an intensity distribution is constructed of the sum of the number of photons with various times through the sample. A measurement of the photon time distribution effectively probes a series of pathlengths through the sample, which is related to absorption and scattering properties of the tissue. Frequency domain or intensity modulated techniques also use a laser as source except the intensity is modulated at radio frequencies with measurements of the intensity and phase made through the tissue sample. Knowledge of the phase shift and the modulation frequency can be used to determine the mean photon pathlength through the tissue sample. Photon time and frequency responses inherently contain information on the optical properties of the tissue. Applying the results from either technique to a modified Beer-Lambert relationship provides a means to reduce or correct for the scatter contributions in the multi-wavelength responses applied to tissue viability, health and status. Time and frequency resolved techniques are used as a correction methodology which use absorption and scattering determined from time or frequency resolved distributions to reduce the spectral variations resulting from the medium.

In some embodiments, the light source may be a laser, for example, an ultra-short pulse solid state, semiconductor laser or a solid state or semiconductor laser output capable of being modulated.

In some embodiments, the detector may have ultra-fast rise and fall times and be capable of detecting low light levels. These may include, for example, photomultipliers (PMT), microchannel plate PMT, streak cameras, photodiodes, PIN photodiodes, and avalanche photodiodes.

In some embodiments, the collector may include, for example, mirrors, fiber optics, and OPR to direct the light to the tissue surface and detector.

In some embodiments, the analyzer may include, for example, electronics to detect, amplify, acquire, and process the responses from the laser and detector to a meaningful result to be used to correct for scattering While the preferred embodiments of the invention have been described above, it will be recognized and understood that various modifications may be made therein, and the appended claims are intended to cover all such modifications which may fall within the spirit and scope of the invention.

1. A device for single or multiple point spectroscopy for determining status of a tissue portion at the surface of the tissue comprising:
a light source emitting energy in the wavelength region between 400-2500 nm;
at least one illuminator delivering light from the light source to the tissue surface;
at least one collector receiving remitted light from the tissue surface;
a detector measuring wavelength data from the remitted light;
an analyzer analyzing the wavelength data from the detector for measuring tissue viability; and
a display unit displaying results from the analyzer.

2. The device according to claim 1 including a wavelength sensitive element for dispersing the collected remitted light into wavelength dependent components.

3. The device according to claim 1 including at least one optical path router for switching between single and multiple illuminators and collectors.

4. The device according to claim 3 wherein an optical path router is connected to the detector for switching between a single collector at a single tissue site and multiple collectors detecting several tissue sites.

5. The device according to claim 3 wherein an optical path router is connected to the light source for switching between a single illuminator illuminating one tissue site and multiple illuminators illuminating several tissue sites.

6. The device according to claim 4 wherein an optical path router is connected to the light source for switching between a single illuminator illuminating one tissue site and multiple illuminators illuminating several tissue sites.

7. The device according to claim 3 or 4 wherein an optical path router is connected to the detector for switching to obtain a sample of the illumination source.

8. The device according to claim 3 including an optical path router for switching between single and multiple illuminators and collectors to monitor the light source.

9. The device according to claim 3 or 4 including an optical path router is connected to the detector for switching to obtain a sample of the illumination source passing through a wavelength calibration standard.

10. The device according to claim 3 or 4 including an optical path router or shutter connected to the detector for switching to obtain a dark spectrum when no light is transmitted.

11. The device according to any one of claims 3 to 5 wherein an optical path router or shutter is connected to the illumination source for switching the transmission of source illumination off.

12. The device according to claim 1 wherein the collector and the illuminator are at a fixed distance relative one another for determining optical depth.

13. The device according to claim 1 wherein the detector includes a two dimensional detector.

14. A device for imaging spectroscopic analysis of a tissue portion comprising:
a light source emitting energy in the wavelength region between 400-2500 nm;
an illuminator delivering light to the tissue portion;
a collector receiving remitted light from the tissue portion;
a detector having a two dimensional sensor array for acquiring images at selected wavelengths from the remitted light;
imaging devices detecting wavelength-dependent images from the detector;
an analyzer processing the images into parameters for assessing tissue status; and
a display unit for displaying the parameters.

15. The device according to claim 14 including an optical path router mounted to the collector for receiving remitted light from multiple sites within the tissue portion.

16. A device for multiple point spectroscopic analysis of a tissue portion comprising:
a light source emitting energy in the wavelength region between 400-2500 nm;
a probe head having mounted thereon:
an illuminator illuminating the tissue portion;
collectors gathering remitted light from the tissue portion, each of said collectors being mounted on the probe head at a position distal to the illuminator and one another for acquiring spectral information at a given tissue depth;
a plurality of detectors dispersing the remitted light gathered by the collectors into wavelength dependent components, each detector being linked to a respective one of the collectors;
an analyzer processing the wavelength dependent components into parameters for assessing tissue status; and
a display unit for showing the parameters.

17. The device according to claim 16 wherein the light source is modulated.

18. The device according to claim 16 wherein the detectors detect the remitted light in the time or frequency domain as the light source modulates.
